(19) United States
(12) Patent Application Publication
(10) Pub. No.: US 2004/0158939 A1
(43) Pub. Date: Aug. 19, 2004

(54) Phase-separated rinse-off hair coloring/conditioning products

(75) Inventors: Robert Lee Wells, Cincinnati, OH (US); Elizabeth Ann Crane, Martinez, CA (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/774,765

(22) Filed: Feb. 9, 2004

Related U.S. Application Data
(63) Continuation of application No. 09/551,036, filed on Apr. 17, 2000, now abandoned.

(51) Int. Cl. A61K 7/13

(52) U.S. Cl. 8/406

(57) ABSTRACT

Phase-separated rinse-off hair coloring/conditioning compositions comprise water soluble dye materials, conditioning materials, and water, wherein the water soluble dyes are concentrated in a discontinuous phase of liquid emulsion droplets dispersed within the droplets to the extent that the intensity of the color imparted by the dye materials to the droplets is greater than the intensity of the color imparted by the dye materials to continuous aqueous phase. The dispersed phase may be formed by the combination of the conditioning materials and the dye materials. Methods of coloring and conditioning hair are also disclosed.

### CROSS REFERENCE TO RELATED APPLICATION

The application is a continuation of application Ser. No. 09/551,036 (Case 8049) filed on Apr. 17, 2000.

### FIELD OF THE INVENTION

The present invention relates to phase-separated rinse-off hair coloring/conditioning compositions and to methods of coloring and conditioning hair therewith.

### BACKGROUND OF THE INVENTION

This invention relates to compositions for imparting color to hair via rinse-off hair conditioning products.

There are generally two ways to deliver color and conditioning benefits to hair: oxidative dye compositions which contain an oxidative dye intermediate and an oxidizing agent, packaged separately from each other, and single product rinse compositions.

Oxidative dye compositions which impart a permanent or semi-permanent color to hair are notorious for leaving the hair damaged. Conditioners have been applied either after the dyeing process or as part of the dye composition, but they have not been able to completely repair the damage caused by the dyeing process.

Soluble direct dyes have been used in several conditioner formulations. Such formulations are non-damaging, but they do not deposit sufficiently to deliver desired levels of color. Although many conditioners are multiphase systems containing fatty alcohol gel networks, liquid crystalline phases, or emulsions, none of these are formulated to concentrate the dye in a separated liquid phase to improve deposition and hair coloring performance in the manner of the present invention.

Accordingly, there is a need for a one-step hair coloring product with conditioning properties which delivers good hair color without damaging the hair.

It has now been found that coloring actives which are a part of separated liquid phases are more efficiently deposited on hair. With water soluble actives, phase separation can be accomplished via coacervation. By selecting specific types of surfactants, coacervates of anionic dyes can be formed in conditioners. In addition, it has been found that certain dyes will partition preferentially into separated aqueous phases created by the conditioning materials.

In view of the foregoing, it is an object of this invention to provide a rinse-off conditioner which imparts increased color to hair. It is a further object of this invention to deliver color without damaging the hair. It is another object of this invention to provide a method of coloring hair concurrently with a conditioning operation.

### BACKGROUND ART

The following art relate to rinse-off hair coloring/conditioning compositions: U.S. Pat. No. 4,975,092, Chan et al., Dec. 4, 1990; EP 0146350 A2, Unilever PLC, 26.06.85; EP 0312343 A3, Unilever PLC, 19.04.89; U.S. Pat. No. 3,930,792, Alperin et al., Jan. 6, 1976; U.S. Pat. No. 5,376,146, Casperson et al., Dec. 27, 1994; U.S. Pat. No. 5,393,305, Cohen et al., Feb. 28, 1995; DE 3712005 A1, L'Oreal, Oct. 22, 1987; DD 271219 A3, VEB Chemisches Werk Miltitz, Aug. 30, 1989 and U.S. Pat. No. 5,356,438, Kim et al., Oct. 18, 1994.

### SUMMARY OF THE INVENTION

In one embodiment, the present invention relates to phase-separated rinse-off hair coloring/conditioning compositions, comprising:

a.) from about 0.1% to about 10% by weight of water-soluble dye materials comprising a member selected from the group consisting of anionic dye, nonionic dye, and mixtures thereof;

b.) from about 0.5% to about 20% by weight of conditioning materials comprising cationic surfactant;

c.) from about 70% to about 99.4% by weight of water;

said water-soluble dye materials being concentrated in a phase of liquid emulsion droplets, said phase being dispersed within a continuous aqueous phase, and wherein said dye materials are concentrated within said droplets to the extent that the intensity of the color imparted by the dye materials to the droplets is greater than the intensity of the color imparted by the dye materials to the continuous aqueous phase. The dispersed phase may be formed by the combination of the conditioning materials and the water-soluble dye materials.

In another embodiment, the conditioning materials of the composition set forth above form droplets of a separated phase and the dye materials preferentially partition into this phase. Again, in this embodiment, the dye materials are concentrated in the phase-separated droplets.

The present invention further relates to methods of simultaneously coloring and conditioning hair.

### DETAILED DESCRIPTION OF THE INVENTION

The present invention is based on the discovery that improved hair coloring can be achieved using direct dyes that are concentrated in a separated phase or coacervate within an otherwise water-based product. This is achieved by using water-soluble dyes which preferentially partition into and/or reside in the separated phase or coacervate in reference over the aqueous phase of the composition.

Theoretically, it may be possible to calculate or otherwise predict the formation of separated phases and/or coacervates that are useful based on the structures of the water-soluble conditioner and other materials in the systems. However, such theoretical predictions are insufficient in the present circumstances wherein the dye molecules become involved with the conditioner and modify its physical characteristics. Fortunately, however, the selection of proper combinations of dye/conditioner/water can be made without undue experimentation by means of visual observations and by using simple light microscopy. Under microscopic examination of the present compositions at 20° C.-30° C., the dye is seen to be concentrated in droplets having an average size in the range from about 0.05 μm to about 100 μm. In preferred compositions, the average size of the droplets ranges from about 0.2 μm to about 50 μm with no droplets being larger than about 100 μm. Excessively large droplets would lead to uneven coloring of the hair shaft.

In the present compositions, the dye molecules must remain concentrated in the droplets which comprise the dispersed phase or in the coacervate. Accordingly, it is necessary to ensure that the dyes not unduly migrate into the aqueous phase. Therefore, the aqueous phase preferably contains no more than about 1%, by weight of the composition, of water-soluble organic solvents such as methanol, ethanol, isopropanol, butanol, benzyl alcohol, hexylene glycol, carbitol, glycerol, polyols, glycols, other C₃-C₉ solvent alcohols and the like. Longer-chain alcohols, e.g., cetyl and stearyl, which are neither water-soluble and nor particularly good solvents and can be used at levels up to about 7% without disrupting the system.

The rinse-off coloring/conditioning compositions of the present invention comprise the essential ingredients, as well any of the additional or optional ingredients described hereinafter. All percentages, parts and ratios are based on the total weight of the compositions of the present invention, unless otherwise specified. All such weights as they pertain to listed ingredients are based on the active level and, therefore, do not include carriers or by-products that may be included in commercially available materials, unless otherwise specified.

The term, “phase separated” or “separated phase”, as used herein, means the formation of two thermodynamically stable liquid phases which exist, not as distinct bulk layers, but as a stable emulsion comprising droplets of one phase dispersed in another phase.

The term “visually distinct”, as used herein, refers to droplets suspended in a continuous phase such that the droplets or droplet phases are visually separate and distinct from the continuous phase when viewed with the aid of a light microscope under 100× to 620× magnification. Such droplets may be present in the composition as manufactured or may form spontaneously from two separated layers when the composition is shaken by hand (i.e., “shake well before using”) immediately prior to application to hair.

The term “water soluble”, as used herein, refers to any material that is sufficiently soluble in water to form a substantially clear solution to the naked eye at a concentration of at least 0.2% by weight of the material in water at 25° C.

The term “dispersed phase”, as used herein, refers to discrete droplets of a composition different from that of the continuous phase. The “continuous phase” is the phase in which any point may be reached from any other point without passing through another phase.

The term “aqueous phase”, as used herein, refers to a phase in which water is the primary carrier.

The term “liquid crystal phase”, as used herein, refers to a phase which is ordered along only one or two orthogonal directions and disordered along the other directions.

The term “coacervation”, as used herein, refers to the separation of a solution into two immiscible liquid phases: a dense coacervate phase which is relatively concentrated in the species causing separation; and a dilute equilibrium phase. Coacervate formation is dependent upon a variety of criteria such as molecular weight, concentration, ratio of interacting ionic materials, ionic strength (including modification of ionic strength, for example, by addition of salts), charge density of the cationic and anionic species, pH, and temperature. Coacervate systems and the effect of these parameters have been described, for example, by J. Caelles, et al., “Anionic and Cationic Compounds in Mixed Systems”, Cosmetics & Toiletries, Vol. 106, April 1991, pp 49-54, C. J. van Oss, “Coacervation, Complex-Coacervation and Flocculation”, J. Dispersion Science and Technology, Vol. 9 (5,6), 1988-89, pp 561-573, and D. J. Burgess, “Practical Analysis of Complex Coacervate Systems”, J. of Colloid and Interface Science, Vol. 140, No. 1, November 1990, pp 227-238, which descriptions are incorporated herein by reference.

Techniques for analyzing complex coacervates are known in the art. For example, microscopic analyses of the rinse-off hair coloring/conditioning compositions of the present invention, neat or at any chosen stage of dilution, can be utilized to identify whether a coacervate phase has formed. Such coacervate phase will be identifiable as an emulsified phase in the composition. The presence of dyes can aid in distinguishing the coacervate phase from other insoluble phases dispersed in the composition because the dye enhances the color difference thereby allowing the droplets to be easily seen.

The term “effective amount”, as used herein, with respect to coloring, refers to the amount necessary to impart the desired amount of color to hair under the usage conditions, and with respect to conditioning, refers to the amount necessary to condition hair under the usage conditions.

The term “substantially free of”, as used herein, refers to the level of the ingredient as being 0.01% or less of the composition.

### Components

The compositions of the present invention comprise as essential components suitable water-soluble dye materials, conditioning materials, and water as the carrier medium.

### The Water-Soluble Dye Materials

An essential component of the present invention is the water-soluble dye materials. The water-soluble dyes are preferably direct dyes. Direct dyes are commonly used in so-called “temporary” hair coloring products and in hair coloring products that are designed to gradually build-up or change the hair color. Direct dyes, in contrast to reactive dyes, do not undergo a chemical reaction in order to deliver color to the hair.

The water-soluble dye, oppositely charged from the conditioning materials, forms a coacervate with the surfactant of the conditioning materials. The water-soluble direct dyes that form a coacervate with cationic surfactant of the conditioning materials are the anionic dyes. The cationic, nonionic, and amine functional dyes, at pH's in which they are substantially cationic, do not form a coacervate; however, they do preferentially partition into the separate aqueous phase formed by the conditioning materials component. The concentration of the dye into the separated phase is visually distinct, and can be confirmed by microscopic observation of the greater color intensity in the droplets than in the continuous phase.

The concentration of the water-soluble dye component in the composition generally ranges from about 0.1% to about 10%, by weight, preferably from about 0.2% to about 5%, by weight, of the composition.

Nonlimiting examples of suitable anionic dyes are FD&C Blue No.1 (CAS number 3844-45-9); FD&C Yellow No.6 (CAS number 2783-94-0); FD&C Yellow No.5 (CAS number 1934-21-0); FD&C Red No.4 (CAS number 4548-53-2); FD&C Red No.40 (CAS number 25956-17-6). D&C Brown No.1 (CAS number 1320-7-6); D&C Green No.5 (CAS number 4403-90-1), and mixtures thereof, although this list is by no means comprehensive. (CAS number is the number assigned to a dye in the Chemical Abstract Service Registry.)

Nonlimiting examples of suitable nonionic dyes are D&C Violet No.2 (CAS number 81-48-1); D&C Yellow No.7 (CAS number 2321-07-5); and D&C Red No.36 (CAS number 2814-77-9), and mixtures thereof.

Nonlimiting examples of suitable cationic dyes are Arianor® Steel Blue (Basic Blue No.99, CI number 56059); Arianor® Madder Red (Basic Red No.76, CI number 12245); Arianor® Mahogany (Basic Brown No.16, CI number 12550); Arianor® Straw Yellow (Basic Yellow No.57, CI number 12719); Basic Yellow No. 11 (CI number 48055); Basic Red No.29 (CI number 11460); Basic Red No.12 (CI number 48070); Basic Blue No.12 (CI number 51180); Basic Blue No.3 (CI number 51104); Basic Violet No.3 (CI number 42555); Basic Violet No.16 (CI number 48013); Basic Green No.4 (CI number 42000); and Arianor® Sienna Brown (Basic Brown No.17, CI number 12251), and mixtures thereof. (CI number is the number assigned to a dye in the Colour Index, 3^{rd} ed., Society of Dyers and Colourists, England, 1971.)

Nonlimiting examples of suitable amine functional dyes are HC Blue No.2 (CAS number 33229-344); HC Blue No.11 (CAS number 23920-15-2); HC Brown No.1 (CAS number 83803-98-9); HC Green No.1 (CAS number 52136-25-1); HC Red No.3 (CAS number 2871-01-4); HC Red No.8 (CAS number 13556-29-1); HC Violet No.2 (CAS number n/a); and HC Yellow No.4 (CAS number 5982043-8), and mixtures thereof.

Other suitable direct dyes may be found in the International Cosmetic Ingredient Dictionary and Handbook, Seventh Edition, 1997.

The preferred dyes for coacervate formation with the conditioning materials of the present invention are the anionic dyes listed above.

### The Conditioning Materials

The rinse-off coloring/conditioning compositions of the present invention also comprise a conditioning materials component to form the coacervate or the liquid crystals with the dye, and provide conditioning performance to the composition. The conditioning component comprises one or more cationic surfactants, and is substantially free of anionic and nonionic surfactants. This cationic surfactant is the primary conditioning agent and, along with other components, provides hair conditioning benefits such as anti-static, soft feel and ease of combing. Such a surfactant should be physically and chemically compatible with the essential components described herein, and should not otherwise unduly impair product stability, aesthetics or performance.

The concentration of the conditioning component in the rinse-off coloring/conditioning composition should be sufficient to provide the desired conditioning performance, and generally ranges from about 0.5% to about 20%, preferably from about 1% to about 10%, by weight, of the composition.

The suitable cationic surfactant for use in the rinse-off hair coloring/conditioning compositions of the present invention comprise select quaternary ammonium or protonated amino compounds. The select surfactants are cationic and must be positively charged at the pH of the conditioner compositions. Generally, the pH of the conditioner composition will be less than 10, typically from about 3 to about 9, preferably from about 4 to about 8, measured neat.

Cationic surfactants useful in the present compositions contain amino or quaternary ammonium hydrophilic moieties which are positively charged when dissolved in water. Cationic surfactants among those useful herein are disclosed in the following documents, all incorporated by reference herein: McCutcheon's, Emulsifiers & Detergents, (1989, published by the M.C. Publishing Company) Schwartz, et al., Surface Active Agents, Their Chemistry and Technology, New York: Interscience Publishers, 1949; U.S. Pat. No. 3,155,591, Hilfer, issued Nov. 3, 1964; U.S. Pat. No. 3,929,678, Laughlin, et al., issued Dec. 30, 1975; U.S. Pat. No. 3,959,461, Bailey, et al., issued May 25, 1976; and U.S. Pat. No. 4,387,090, Bolich, Jr., issued Jun. 7, 1983.

Among the quaternary ammonium-containing cationic surfactant materials useful herein are those of the general formula:

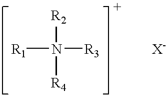

wherein R₁-R₄ are independently an aliphatic group of from about 1 to about 22 carbon atoms, or an aromatic, alkoxy, polyozyalkylene, alkylamido, hydroxyalkyl, aryl or alkylaryl group having from about 12 to about 22 carbon atoms; and X is an anion selected from halogen, acetate, phosphate, nitrate and alkylsulfate radicals. The aliphatic groups may contain, in addition to carbon and hydrogen atoms, either linkages, and other groups such as amino groups.

Other quaternary ammonium salts useful herein have the formula:

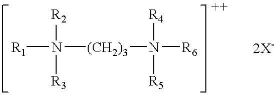

wherein R₁ is an aliphatic group having from about 16 to about 22 carbon atoms, R₂, R₃, R₄, R₅, and R₆ are selected from hydrogen and alkyl having from about 1 to about 4 carbon atoms, and X is an ion selected from halogen, acetate, phosphate, nitrate and alkyl sulfate radicals. Such quaternary ammonium salts include tallow propane diammonium dichloride.

Preferred quaternary ammonium salts include dialkyldimethyl-and dialkyldimethyl-benzyl substituted ammonium chlorides, wherein one or two alkyl groups have from about 12 to about 22 carbon atoms and are derived from long-chain fatty acids, such as hydrogenated tallow fatty acid (tallow fatty acids yield quaternary compounds wherein R₁ and R₂ have predominately from 16 to 18 carbon atoms). Examples of quaternary ammonium salts useful in the present invention include ditallowdimethyl ammonium chloride, ditallowdimethyl ammonium methyl sulfate, dihexadecyl dimethyl ammonium chloride, di(hydrogenated tallow) dimethyl ammonium chloride, dioctadecyl dimethyl ammonium chloride, dieicosyl dimethyl ammonium chloride, didocoysl dimethyl ammonium chloride, di(hydrogentated tallow) dimethyl ammonium acetate, dihexadecyl dimethyl ammonium chloride, dihexadecyl dimethyl ammonium acetate, ditallow dipropyl ammonium phosphate, ditallow dimethyl ammonium nitrate, di(coconutalkyl) dimethyl ammonium chloride, and stearyl dimethyl benzyl ammonium chloride. Ditallow dimethyl ammonium chloride, dicetyl dimethyl ammonium chloride, stearyl dimethyl benzyl ammonium chloride and cetyl trimethyl ammonium chloride are preferred quaternary ammonium salts useful herein. Di-(hydrogenated tallow) dimethyl ammonium chloride is a particularly preferred quaternary ammonium salt.

Salts of primary, secondary and tertiary fatty amines are also preferred cationic surfactant materials. The alkyl groups of such amines preferably have from about 12 to about 22 carbon atoms, and may be substituted or unsubstituted. Secondary and tertiary amines are preferred, tertiary amines are particularly preferred. Useful amines include stearamido propyl dimethyl amine, diethyl amino ethyl stearamide, dimethyl stearamine, dimethyl soyamine, soyamine, myristyl amine, tirdecyl amine, ethyl stearylamine, N-taliowpropane diamine, ethoxylated (5 moles E.O.) stearylamine, dihydroxy ethyl stearylamine, and arachidylbehenylamine. Suitable amine salts include the halide, acetate, phosphate, nitrate, citrate and lactate salts. Such salts include stearylamine hydrochloride, soyamine chloride, stearylamine acetate or formate, N-tallowpropane diamine dichloride and stearamidopropyl dimethylamine citrate. Cationic amine surfactants included among those useful in the present invention are disclosed in U.S. Pat. No. 4,275,055, Nachtigal, et al., issued Jun. 23, 1981, incorporated by reference herein.

The concentration of the cationic surfactant in the conditioning materials component generally ranges from about 50% to about 100%, preferably from about 75% to about 100.

The weight ratio of dye materials to total conditioning materials is generally in the range from about 1:200 to about 20:1, preferably from about 1:5 to about 5:1.

### Water

The conditioner compositions of the present invention are aqueous systems which comprise from about 70% to about 99.4%, preferably from about 75% to about 95%, more preferably from about 80% to about 90%, water by weight of the composition, as the carrier.

### Preferred Optional Components

### Additional Conditioning Agents

From about 0% to about 10% of the conditioner compositions of the present invention may include additional conditioning agents which are non-cationic. These conditioning agents render hair somewhat hydrophobic and they do not hinder deposition of the dye coacervate onto the hair. These conditioning agents generally comprise one or more lipid materials. These lipids provide additional conditioning benefits and also, in combination with the cationic surfactant(s) form a gel network thickened vehicle. Gel-type vehicles provide for shear thinning rheology and Gel-type vehicles are generally described in the following documents, all incorporated by reference herein. Barry, “The Self Bodying Action of the Mixed Emulsifier Sodium Dodenyl Sulfate/Cetyl Alcohol”, 28 J. of Colloid and Interface Science, 82-91 (1968); Barry et al., “The Self-Bodying Action of Alkyltrimethylammonium Bromides/Cetostearyl Chain Length”, 35 J. of Colloid and Interface Science, 689-708 (1971); Barry et al., “Rheology of Systems Containing Cetomacrogol 1000-Cetostearyl Alcohol, I. Self Bodying Action”, 38 J. of Colloid and Interface Science, 616-625 (1972).

The lipid materials are essentially water-insoluble, and contain hydrophobic and hydrophilic moieties. Lipid materials include naturally or synthetically-derived acids, alcohols, esters, ethers, ketones and non-surfactant amides with carbon chains of from about 12 to about 22, preferably from 16 to about 18, carbon atoms in length. Fatty alcohols and fatty esters are preferred; fatty alcohols are particularly preferred.

Lipid materials among those useful herein are disclosed in Bailey's Industrial Oil and Fat Products, (3^{rd} edition., D. Swern, ed., 1979), incorporated by reference herein. Fatty alcohols included among those useful herein are disclosed in the following documents, all incorporated by reference herein: U.S. Pat. No. 3,155,591, Hilfer issued Nov. 3, 1964; U.S. Pat. No. 4,165,369, Watanabe, et al., issued Aug. 21, 1979; U.S. Pat. No. 4,269,824, Villamarin, et al., issued May 26, 1981; British Specification 1,532,585, published Nov. 15, 1978; and Fukushima. et al., “The Effect of Cetostearyl Alcohol in Cosmetic Emulsions”, 98 Cosmetics & Toiletries 89-112 (1983). Fatty esters included among those useful herein are disclosed in U.S. Pat. No. 3,341, Kaufman, et al., issued Sep. 12, 1976 (incorporated by reference herein).

Preferred esters for use herein include cetyl palmitate and glycerylmonostearate. Cetyl alcohol and stearyl alcohol are preferred alcohols. A particularly preferred lipid material is comprised of a mixture of cetyl alcohol and stearyl alcohol containing from about 55% to about 65% (by weight of mixture) of cetyl alcohol.

### Silicones

From about 0% to about 5% of the compositions of the present invention may optionally include non-volatile, non-surfactant silicone components as additional hair conditioners. These silicones do not hinder the deposition of the dye coacervate onto the hair. Typically, the silicone components are intermixed into the aqueous coloring compositions, forming a separate, discontinuous silicone phase. The silicone conditioning component will comprise a silicone fluid conditioning agent such as a silicone fluid and can also comprise other ingredients, such as a silicone resin to enhance silicone fluid deposition efficiency or enhance glossiness of the hair (especially when high refractive index (e.g., above about 1.46), silicone conditioning agents are used (e.g., highly phenylated silicones)). Such silicones do not negatively impact dye deposition.

As used herein, “nonvolatile” refers to a silicone material with little or no significant vapor pressure under ambient conditions, as is understood by those in the art. The boiling point under one atmosphere (atm) will preferably be at least about 250° C., more preferably at least about 275° C., most preferably at least about 300° C. The vapor pressure is preferably about 0.2 mm Hg at 25° C. or less, preferably about 0.1 mm Hg at 25° C. or less.

The silicone conditioning agent phase may contain some volatile silicone, nonvolatile silicone, or mixtures thereof. Typically, if volatile silicones are present, it will be due to their use as a solvent or carrier for commercially available forms of nonvolatile silicone materials ingredients, such as silicone gums and resins.

The silicone conditioning agents for use in the compositions of the present invention preferably have a viscosity of from about 20 to about 2,000,000 centistokes, more preferably from about 1,000 to about 1,800,000 centistokes, even more preferably from about 10,000 to about 1,500,000 centistokes, most preferably from about 30,000 to about 1,000,000 centistokes, at 25° C. The viscosity can be measured by means of a glass capillary viscometer as set forth in Dow Corning Corporate Test Method CTM0004, Jul. 20, 1970.

Optional silicone fluids for use in the present compositions include silicone oils which are flowable silicone materials with a viscosity of less than 1,000,000 centistokes, preferably between about 5 and 1,000,000 centistokes, more preferably between about 10 and about 600,000 centistokes, even more preferably between about 10 and about 500,000 centistokes, most preferably between 10 and 300,000 centistokes at 25° C. Suitable silicone oils include polyalkyl siloxanes, polyaryl siloxanes, polyalkylaryl siloxanes, polyether siloxane copolymers, and mixtures thereof. Other insoluble, nonvolatile silicone fluids having conditioning properties can also be used.

The aliphatic or aryl groups substituted on the siloxane chain may have any structure as long as the resulting silicones remain fluid at room temperature, are hydrophobic, are neither irritating, toxic nor otherwise harmful when applied to the hair or skin, are compatible with the other components of the herein described coloring/conditioning compositions, are chemically stable under normal use and storage conditions, are insoluble in the compositions of the present invention, and are capable of being deposited on and, of conditioning, the hair.

Preferred alkyl and alkenyl substituents are C₁-C₅ alkyls and alkenyls, more preferably C₁-C₄, most preferably C₁-C₂. The aliphatic portions of other alkyl-, alkenyl-, or alkynyl-containing groups (such as alkoxy, alkaryl, and alkamino) can be straight or branched chains and preferably have from one to five carbon atoms, more preferably from one to four carbon atoms, even more preferably from one to three carbon atoms, most preferably from one to two carbon atoms. As discussed above, these substituents can also contain amino functionalities, e.g. alkamino groups, which can be primary, secondary or tertiary amines or quaternary ammonium. These include mono-, di- and tri- alkylamino and alkoxyamino groups wherein the aliphatic portion chain length is preferably as described above. The R substituents can also be substituted with other groups, such as halogens (e.g., chloride, fluoride, and bromide), halogenated aliphatic or aryl groups, and hydroxy (e.g., hydroxy substituted aliphatic groups). Suitable halogenated groups include, for example, tri-halogenated (preferably fluoro) alkyl groups.

Suitable substituent groups include methyl, ethyl, propyl, phenyl, methylphenyl and phenylmethyl. The preferred silicones are polydimethyl siloxane, polydiethylsiloxane, and polymethylphenylsiloxane. Polydimethyl-siloxane is especially preferred. Other suitable groups include methyl, methoxy, ethyl, ethoxy, propyl, propoxy, aryl and aryloxy.

The nonvolatile polyalkylsiloxane fluids that may be used include, for example, polydimethylsiloxanes. These siloxanes are available, for example, from the General Electric Company in their Viscasil R and SF 96 series, and from Dow Corning in their Dow Corning 200 series.

The polyalkylaryl siloxane fluids that may be used, also include, for example, polymethylphenylsiloxanes. These siloxanes are available, for example, from the General Electric Company as SF 1075 methyl phenyl fluid or from Dow Corning as 556 Cosmetic Grade Fluid.

The polyether siloxane copolymers that may be used include, for example, a polypropylene oxide modified polydimethylsiloxane (e.g., Dow Corning DC-1248) although ethylene oxide or mixtures of ethylene oxide and propylene oxide may also be used. The ethylene oxide and polypropylene oxide level must be sufficiently low to prevent solubility in water and the composition hereof.

Other suitable silicone fluids for use as a component of the silicone conditioning agents are insoluble silicone gums. These gums are polyorganosiloxane materials having a viscosity at 25° C. of greater than or equal to 1,000,000 centistokes. Silicone gums are described in U.S. Pat. No. 4,152,416; Noll and Walter, Chemistry and Technology of Silicones, New York: Academic Press 1968; and in General Electric Silicone Rubber Product Data Sheets SE 30, SE 33, SE 54 and SE 76, all of which are incorporated herein by reference. The silicone gums will typically have a mass molecular weight in excess of about 200,000, generally between about 200,000 and about 1,000,000, specific examples of which include polydimethylsiloxane, (polydimethylsiloxane) (methylvinylsiloxane) copolymer, poly(dimethylsiloxane) (diphenyl siloxane) (methylvinylsiloxane) copolymer, and mixtures thereof.

The silicone conditioning agent can also comprise a mixture of polydimethylsiloxane gum (viscosity greater than about 1,000,000 centistokes) and polydimethylsiloxane oil (viscosity from about 10 to about 100,000 centistokes), wherein the ratio of gum to fluid is from about 30:70 to about 70:30, preferably from about 40:60 to about 60:40.

The silicone conditioning agent can also comprise a mixture of volatile silicone such as D5 cyclomethicones and gums in a ratio of D5 to gum of 60:40 to 95:5, preferably 80:20 to 90:10.

References disclosing examples of some suitable silicone fluids for use in the coloring/conditioning compositions include U.S. Pat. No. 2,826,551, U.S. Pat. No. 3,964,500, U.S. Pat. No. 4,364,837, British Patent 849,433, and Silicon Compounds, Petrarch Systems, Inc. (1984), all of which are incorporated herein by reference.

Silicone resins can be included in the silicone conditioning agent. These resins are highly crosslinked polymeric siloxane systems. The crosslinking is introduced through the incorporation of trifunctional and tetrafunctional silanes with monofunctional or difunctional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units (and hence, a sufficient level of crosslinking) such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinyl-chlorosilanes, and tetrachlorosilane, with the methyl-substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art.

Background material on silicones including sections discussing silicone fluids, gums, and resins, as well as manufacture of silicones, can be found in Encyclopedia of Polymer Science and Engineering, Volume 15, Second Edition, pp. 204-308, John Wiley & Sons, Inc., 1989, incorporated herein by reference.

Silicone materials and silicone resins in particular, can conveniently be identified according to a shorthand nomenclature system well known to those skilled in the art as “MDTQ” nomenclature. Under this system, the silicone is described according to presence of various siloxane monomer units which make up the silicone. Briefly, the symbol M denotes the monofunctional unit (CH₃)₃SiO₅; D denotes the difunctional unit (CH₃)₂SiO; T denotes the trifunctional unit (CH₃)SiO_{1.5}; and Q denotes the quadri- or tetra-functional unit SiO₂. Primes of the unit symbols, e.g., M′, D′, T′, and Q′ denote substituents other than methyl, and must be specifically defined for each occurrence. Typical alternate substituents include groups such as vinyl, phenyls, amines, hydroxyls, etc. The molar ratios of the various units, either in terms of subscripts to the symbols indicating the total number of each type of unit in the silicone (or an average thereof) or as specifically indicated ratios in combination with molecular weight complete the description of the silicone material under the MDTQ system. Higher relative molar amounts of T, Q, T′ and/or Q′ to D, D′, M and/or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MDT and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000.

The weight ratio of the nonvolatile silicone fluid, having refractive index below 1.46, to the silicone resin component, when used, is preferably from about 4:1 to about 400:1, preferably this ratio is from about 9:1 to about 200:1, more preferably from about 19:1 to about 100:1, particularly when the silicone fluid component is a polydimethylsiloxane fluid or a mixture of polydimethylsiloxane fluid and polydimethylsiloxane gum as described above. Insofar as the silicone resin forms a part of the same phase in the compositions hereof as the silicone fluid, the sum of the fluid and resin should be included in determining the level of silicone conditioning agent in the composition.

The number average particle size of the optional silicone component can vary widely depending on the formulation and/or the desired characteristics. Number average particle sizes preferred for use in the present invention range from about 10 nanometers to about 100 microns, more preferably from about 30 nanometers to about 20 microns.

### Other Optional Components

In addition to the essential and other components described hereinbefore, the rinse-off coloring/conditioning compositions of the present invention may further comprise one or more optional components known for use is hair care or personal care products, provided that the optional components are physically and chemically compatible with the essential components describes herein, or do not otherwise unduly impair product stability, aesthetics or performance. These other optional components may comprise, in total, from about 0% to about 10% of the composition

Nonlimiting examples of such optional components are disclosed in International Cosmetic Ingredient Dictionary and Handbook, Seventh Edition, 1997 which is incorporated by reference herein in its entirety. Some nonlimiting examples of such optional components are styling agents; perfumes; preservatives (e.g., benzoic acid, methyl paraben, propyl paraben, imidazolidinyl urea, iodopropynyl butyl carbamate, methylisothiazolinone, methylchloroisothiazolinone); noncationic anti-static agents; anti-dandruff agents (e.g., pyridinethione salts and selenium compounds); viscosity adjusting agents; thickeners; pH adjusting agents (e.g., sodium citrate, citric acid, succinic acid, phosphoric acid, sodium hydroxide, and sodium carbonate); anti-microbial agents (e.g., Triclosans® or triclocarbon); anti-oxidants; diluents; pearlescent aids; proteins; scalp sensates (e.g., menthol); topical anesthetics; skin active agents; sunscreening agents; vitamins; humectants; pediculocides (e.g., pyrethrins) and mixtures thereof.

### Method of Manufacture

The compositions of the present invention, in general, can be made by heating all ingredients to about 73° C., mixing well, and then cooling to room temperature.

### Method of Use

The rinse-off coloring/conditioning compositions of the present invention are used in a conventional way to color and condition hair. An effective amount of the composition is applied to the hair, that has preferably been wetted with water, allowed to remain in contact with the hair and then rinsed-off. Such effective amounts generally range from about 1 g. to about 50 g., preferably from about 1 g. to about 20 g. Application to the hair typically includes working the composition through the hair such that most or all of the hair is contacted with the composition.

This method for coloring and conditioning the hair comprises the steps of: a) wetting the hair with water, b) applying an effective amount of the coloring composition to the hair, c) working the composition throughout the hair, d) rinsing the coloring composition from the hair using water. Working the composition throughout the hair is in accordance to the manner used in a standard conditioning operation. The composition is typically allowed to stand in contact with the hair for about 3 seconds to about 5 minutes before rinsing; however, a longer time period may be used. The hair may be shampooed and rinsed prior to application of the coloring composition, but this is not necessary. These steps can be repeated as many times as desired to achieve the color shade and conditioning benefits desired. The repetition of this process yields a more intense coloration of the hair.

### EXAMPLES

The phase-separated rinse-off hair coloring/conditioning compositions in Examples I-III illustrate specific embodiments of the present invention, but are not intended to be limiting thereof. Other modifications can be undertaken by the skilled artisan without departing from the spirit and scope of this invention. Ingredients are hereinafter identified by chemical, trade or the Cosmetic, Toiletry, and Fragrance Association (CTFA) name.

Example I is made by heating all ingredients to about 73° C., mixing well, and then cooling to room temperature. Example II is made by heating water, Dehyquart® E, cetyl alcohol, stearyl alcohol, ethylenediaminetetraacetic acid (EDTA) and benzyl alcohol to about 73° C. and mixing for about 45 minutes. Then, the mixture is cooled to room temperature and the Kathon® and the dye solution is mixed in. Example III is made by heating the water to 170° F., adding Variquat® 638, cetyl alcohol, stearyl alcohol, EDTA and benzyl alcohol to the water and mixing for 15 b minutes, then cooling the mixture 130° F. Then, the remaining ingredients are added and the mixture is cooled to room temperature.

EXAMPLE I

| Ingredient | Amount (weight percent) |
|---|---|
| Variquat ® 638 (PEG-2 Cocomonium | 2.41 |
| chloride) | |
| Cetyl Alcohol | 2.00 |
| Stearyl Alcohol | 3.60 |
| EDTA (Ethylenediamine tetraaceticacid) | 0.10 |
| Benzyl Alcohol | 0.40 |
| Kathon | 0.057 |
| FD&C Blue No. 1 | 1.00 |
| Water | q.s. |

EXAMPLE II

| Ingredient | Amount (weight percent) |
|---|---|
| Dehyquart ® E (Hydrocetyl hydroxyethyl | 2.00 |
| dimonium chloride) | |
| Cetyl Alcohol | 2.00 |
| Stearyl Alcohol | 3.60 |
| EDTA (Ethylenediamine tetraaceticacid) | 0.10 |
| Benzyl Alcohol | 0.40 |
| Kathon | 0.33 |
| Dye Solution¹ | 8.33 |
| Water | q.s. |

¹Dye solution consists of 88.0% water and 12.0% FD&C Blue No. 1.

EXAMPLE III

| Ingredient | Amount (weight percent) |
|---|---|
| Variquat ® 638 (PEG-2 Cocomonium | 2.41 |
| chloride) | |
| Cetyl Alcohol | 2.00 |
| Stearyl Alcohol | 3.60 |
| EDTA (Ethylenediamine tetraaceticacid) | 0.10 |
| Dimethicone gum/Cyclomethicone | 4.20 |
| mixture¹ | |
| Kathon ® CG² | 0.033 |
| Fragrance | 0.25 |
| Dye Solution³ | 8.33 |
| Water | q.s. |

¹85% dimethicone gum/15% D5 cyclomethicone
²methylchloroisothiazalinone, methylisothiazalinone
³Dye solution consists of 88.0 wt. % water and 12.0 wt. % FD&C Yellow No. 6

The conditioner compositions, illustrated in Examples I-III, provide excellent rinse-off, even coloring, conditioning and mildness, and do not stain or irritate the skin.

The composition of Example I, examined microscopically, shows dark blue droplets about 10 μm to about 100 μm in diameter, uncolored crystals, and light blue continuous phase.

The composition of Example II, examined microscopically, shows aggregates of dark blue droplets. The droplets are about 20 μm to about 40 μm in diameter. The aggregates are about 60 μm to about 300 μm across. The continuous phase has no blue color.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is, therefore, intended to cover in the appended claims all such changes and modifications that are within the scope of this invention.

What is claimed is:

1. A rinse-off hair coloring/conditioning composition, comprising:
a.) from about 0.1% to about 10% by weight of water-soluble dye materials comprising a member selected from the group consisting of anionic dye, nonionic dye, and mixtures thereof;
b.) from about 0.5% to about 20% by weight of conditioning materials comprising cationic surfactant; and
c.) from about 70% to about 99.4% by weight of water;
said water-soluble dye materials being concentrated in a dispersed phase of liquid emulsion droplets dispersed within a continuous aqueous phase; wherein said dye materials are concentrated within said droplets to the extent that the intensity of the color imparted by the dye materials to the droplets is greater than the intensity of the color imparted by the dye materials to the continuous aqueous phase.

2. A rinse-off hair coloring/conditioning composition according to claim 1, wherein the average size of said liquid emulsion droplets are between about 0.05 μm and about 100 μm in diameter.

3. A rinse-off hair coloring/conditioning composition according to claim 1, wherein said water-soluble dye materials are present at a concentration of from about 0.2% to about 5% by weight.

4. A rinse-off hair coloring/conditioning composition according to claim 1, wherein said conditioning materials are present at a concentration of from about 1% to about 10% by weight.

5. A rinse-off hair coloring/conditioning composition according to claim 1, wherein said conditioning materials and said water-soluble dye materials interact to form the dispersed liquid phase in which said water-soluble dye materials are concentrated.

6. A rinse-off hair coloring/conditioning composition according to claim 1, wherein said cationic surfactant conditioning materials consist of quaternary ammonium surfactants.

7. A rinse-off hair coloring/conditioning composition according to claim 6, wherein said quaternary ammonium surfactant is selected from the group comprising of: methyl bis(2-hydroxyethyl) coco-ammonium chloride, methyl bis(2-hydroxyethyl) tallow ammonium chloride, methyl bis(2-hydroxyethyl) oleyl ammonium chloride, cocomethyl bis(2-hydroxyethyl) ammonium chloride, methyl bis(oleylamidoethyl) 2-hydroxyethyl ammonium methyl sulfate, dilauryl acetyl dimonium chloride, hydroxycetyl hydroxyethyl dimonium chloride, cetyl trimonium chloride, and isostearamidopropyl laurylacetodimonium chloride and mixtures thereof.

8. A rinse-off hair coloring/conditioning composition according to claim 5, wherein said water-soluble dye materials consists of direct dyes.

9. A rinse-off hair coloring/conditioning composition according to claim 8, wherein said direct dyes are anionic.

10. A rinse-off hair coloring/conditioning composition according to claim 6 further comprising a fatty alcohol gel network.

11. A rinse-off hair coloring/conditioning composition according to claim 1, wherein said conditioning materials form a separated liquid phase and said water-soluble dye preferentially partitions into this separated aqueous phase, such that, microscopically, the color is more intense in dispersed droplets than in a continuous aqueous phase.

12. A rinse-off hair coloring/conditioning composition according to claim 1 further comprising non-cationic conditioning agents.

13. A rinse-off hair coloring/conditioning composition according to claim 12 wherein said non-cationic agents are silicones.

14. A rinse-off hair coloring/conditioning composition according to claim 1 additionally comprising a member selected from the group consisting of styling agents, perfumes, preservatives, non-cationic anti-static agents, polyethylene glycol, suspending agents, anti-dandruff agents, viscosity adjusting agents and thickeners, pH adjusting agents, anti-microbial agents, anti-oxidants, diluents, pearlescent aids, scalp senates, topical anesthetics, proteins, skin active agents, sunscreening agents, humectants, vitamins, pediculocides, and mixtures thereof.

15. A method of coloring and conditioning hair, comprising:
a) wetting said hair with water;
b) applying to said hair an effective amount of a coloring/conditioning composition according to claim 1; and
c) rinsing said coloring/conditioning composition from said hair using water.

16. A rinse-off hair coloring/conditioning composition according to claim 1, wherein said conditioning materials form a separated aqueous phase and said water-soluble dye preferentially partitions into this separated aqueous phase, such that, microscopically, the color is more intense in dispersed droplets than in a continuous aqueous phase.
